(19) United States
(12) Patent Application Publication
(10) Pub. No.: US 2008/0269597 A1
(43) Pub. Date: Oct. 30, 2008

(54) Gauze or the Like Having a Device for its Detection at a Distance

(76) Inventor: Diego Benetti, Concesio (IT)

Correspondence Address: LUCAS & MERCANTI, LLP, 475 PARK AVENUE SOUTH, 15TH FLOOR, NEW YORK, NY, 10016, US

(21) Appl. No.: 12/089,298

(22) PCT Filed: Aug. 30, 2006
(86) PCT No.: PCT/IB06/02359

§ 371 Date: Apr. 4, 2008

(30) Foreign Application Priority Data
Oct. 7, 2005 (IT) MI2005A001884

(51) Int. Cl. A61B 5/06; A61F 13/00

(52) U.S. Cl. 600/424; 602/41

(57) ABSTRACT

A gauze or other material for surgical use comprising at least one presence detection device for remotely identifying said gauze or other material for surgical use. The presence detection device can be a radio-opaque element or can be activated by a radio-frequency or a magnetic signal. It can be provided with a chip, an antenna and a memory.

### FIELD OF THE INVENTION

The present invention relates to a gauze for surgical use, the presence of which is remotely detectable.

### BACKGROUND ART

Multi-web gauzes made of hydrophilic warp and weft tissue, for example cotton, are known to be normally used in the surgical field for temporarily tamponing any loss of blood or fluids from a patients being subjected to an operation.

It is also known that, at the end of a surgical operation, the risks connected to gauzes that have been forgotten by the surgeon or paramedic inside the patient's body are very high, which lead to severe and/or mortal consequences for the patient.

It should be observed that, while particular reference is made herein and below to a gauze for surgical use, any other material or surgical instrument used in an operating room during a surgical operation, such as bandages, patches, or scalpels, or anything else not mentioned herein, may as well implement the technical solution as claimed herein.

In order to avoid the above-mentioned drawback, measures are taken in operating rooms for counting the gauzes, both those used during a surgical operation and those discarded at the end of the operation, which however may prove to be poorly reliable, as the delicate or urgent operations are often critical.

In order to provide for a correct counting of the gauzes, particular containers are often used, also known as “gauze counter”, which are provided with an automatic counting device that increments the sum by one unit at each introduction of material within the gauze counter.

Unfortunately, it is not uncommon that materials other than gauzes are introduced in the gauze counter, such as patches, envelopes or other, thereby leading to an inaccurate count of the gauzes used during the operation.

In the new-concept gauzes, the warp and weft webs composing the gauze contain threads made of radiopaque material, which allow detecting, through a normal radiological examination, the presence of one or more gauzes that have been forgotten in the patient's body. Said solution, though effective, has various drawbacks. In fact, the radiological examination to be carried out at the end of the operation entails a further trouble for the patient and extra costs for the hospital; furthermore, the radiography resulting from said radiological examination may be often quite difficult to read and even skilled and qualified people may make in gross mistakes.

The object of the present invention is to provide a gauze or other material for surgical use which can be remotely detected, is cost-effective and simple to manufacture.

### SUMMARY OF THE INVENTION

It is therefore an object of the present invention to meet such requirements by providing a gauze or other material for surgical use, wherein at least one presence detection device is comprised for remotely identifying said gauze or said other surgical material.

According to a peculiar aspect of the present invention, said detection device is capable of changing the intensity of a magnetic field, in which it may be immersed. Furthermore, said detection device can be thread-like and interlaced in the warp or weft of said gauze.

### BRIEF DESCRIPTION OF THE DRAWINGS

Further characteristics and advantages of the present invention will be more clearly understood from the detailed description of a preferred, although non exclusive embodiments of the gauze or other material for surgical use such as illustrated in the annexed figures, in which:

FIG. 1 is a view of the gauze according to a first embodiment;

FIG. 2 is a view of the gauze in accordance with a second embodiment of the present invention.

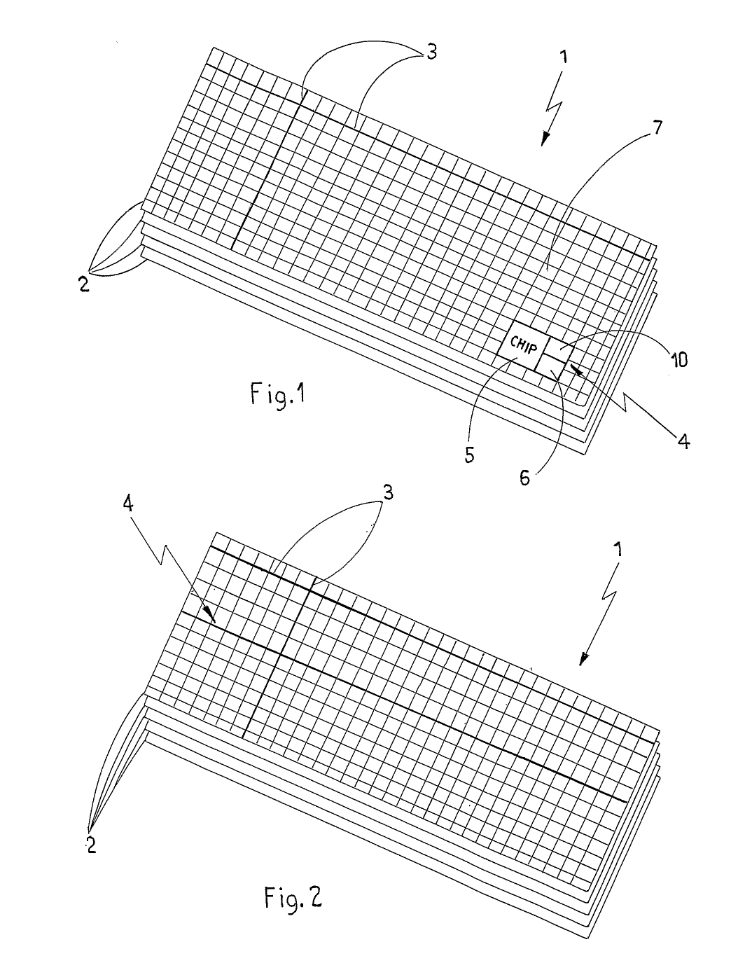

### DETAILED DESCRIPTION OF A PREFERRED EMBODIMENT OF THE INVENTION

With particular reference to these figures, the gauze according to the invention has been generally designated with 1.

The gauze 1 is made of warp and weft hydrophilic tissue, such as cotton, and is provided with webs 2 having a substantially rectangular shape. It should be also observed that said gauze 1 can further comprise a radiopaque thread-like element 3 arranged in the warp and/or weft of the hydrophilic tissue, which is capable of allowing said gauze 1 to be detected through a radiographic examination.

In a first embodiment of the invention, said gauze 1 comprises a presence detection device 4 in a substantially rectangular shape, which is fastened, for example by bonding, to the upper surface 7 of said gauze 1 or between said webs 2 or inserted within a suitable housing pocket (not shown) obtained on said gauze 1. Alternatively, said detection device 4 can be provided with gripping elements (not shown) that can be inserted between the meshes of the warp and weft of said webs 2 thus fastening to said gauze 1.

Said detection device 4 is provided with an integrated electronic circuit 5 (CHIP) and a communication antenna 6 that is capable of receiving and/or transmitting any information in case processed by said electronic circuit 5. When said detection device 4 is subjected to a specific external radio frequency signal, it is activated and transmits, in turn, a specific signal to the emitting source of the radio frequency signal.

Said detection device 4 can further comprise a memory element 10 capable of containing further information, such as the identification number or type or sterility life of the gauze, or other useful information for the medical or paramedical staff either before, during or after the surgical operation.

In a second embodiment of the invention, said detection device 4 is substantially thread-like and is inserted in the warp and/or weft of said gauze 1. Said detection device 4 is made of a material capable of changing the intensity of a magnetic field to which it is subjected, such as, magnetizable or magnetically active metal.

Said gauze 1, in the event it is inadvertedly left in the patient's body during the surgical operation, can be easily identified by means of an external device for signal generation and reception (not shown herein). Said signal-generating and receiving device is placed at a certain distance from the patient undergoing a surgical operation and generates a signal that is recognized by the detection device 4 constrained to said gauze 1, for example of the radio frequency type, as in the first embodiment of the gauze 1, or magnetic type, as in the second embodiment of the gauze 1. Subsequently, this signal-generating and receiving device is capable of receiving a feedback signal from said detection device 4 indicative of the presence of said gauze 1, provided that the latter is within the working range of said signal--generating and receiving device. Said detection devices 4 also operate in the most serious cases, i.e. when said gauze 1 is left in the patient's body even after the surgical operation, in fact said radio or magnetic signal that are generated and received by the detection device and signal-generating and receiving device are capable of passing through the tissues in the human body without being affected by substantial interferences.

Practically, the invention described has been found to achieve the proposed objects.

1. A gauze or other material for surgical use, characterized in that it comprises at least one presence detection device for remotely identifying said gauze or other material for surgical use.

2. The gauze or the like according to claim 1, characterized in that it comprises more warp and weft webs wherein said at least one detection device is fastened to a web or between said webs by bonding or within a pocket obtained on said gauze or at the surface of said gauze by means of gripping elements.

3. The gauze or the like according to claim 1, characterized in that at least one detection device is substantially thread-like and is a part of the warp and/or weft of said webs.

4. The gauze or the like according to claim 1, characterized in that said presence detection device comprises at least one element that is capable of inducing an intensity variation of a magnetic field.

5. The gauze or the like according to claim 1, characterized in that said presence detection device comprises at least one integrated electronic circuit (CHIP) and at least one antenna for said integrated electronic circuit, said at least one antenna being capable of receiving and transmitting radio frequency waves.

6. The gauze or the like according to claim 5, characterized in that said presence detection device comprises a memory element capable of containing useful additional information to be communicated to the outside.

7. The gauze or the like according to claim 1, characterized in that it comprises at least one radiopaque element.
